(19) United States
(12) Patent Application Publication
(10) Pub. No.: US 2021/0251689 A1
(43) Pub. Date: Aug. 19, 2021

(54) MICROWAVE SYSTEM FOR GLOBAL ENDOMETRIAL ABLATION

(71) Applicants: HOLOGIC, INC., Marlborough, MA (US); KANSAS STATE UNIVERSITY RESEARCH FOUNDATION, Manhattan, KS (US)

(72) Inventors: Punit Prakash, Manhattan, KS (US); Hojjatollah Fallahi, Minneapolis, MN (US); Jessica T. Schenck, Shrewsbury, MA (US); Eric A. Frattura, Shrewsbury, MA (US)

(73) Assignees: HOLOGIC, INC., Marlborough, MA (US); KANSAS STATE UNIVERSITY RESEARCH FOUNDATION, Manhattan, KS (US)

(21) Appl. No.: 17/251,592

(22) PCT Filed: Jun. 12, 2019
(86) PCT No.: PCT/US2019/036845

§ 371 Date: Dec. 11, 2020

Related U.S. Application Data
(60) Provisional application No. 62/684,580, filed on Jun. 13, 2018.

(51) Int. Cl. A61B 18/18; A61B 5/0507; A61B 5/00

(52) U.S. Cl. CPC A61B 18/1815; A61B 2018/00559; A61B 5/4836; A61B 5/0507

(57) ABSTRACT

A tissue ablation system includes an antenna, a microwave energy source, measurement apparatus including a signal generator and a detector, a controller, and a switch assembly arranged to alternatively electrically couple the antenna to the microwave energy source or to the measurement apparatus, wherein the controller is configured to cause the switch assembly to alternate between assessment mode and ablation mode, and wherein the controller is configured to control the delivery of ablative microwave energy during an ablation mode based at least in part on a difference between a then-current broadband reflection coefficient spectrum and a previously measured broadband reflection coefficient spectrum obtained by delivery of a spectrum of individual non-ablative microwave signals and detecting the reflection of same during respective assessment modes.

### FIELD

The presently disclosed invention(s) relate generally to medical devices. More particularly, the present disclosure relates to systems and methods for tissue ablation in a body cavity using electromagnetic (microwave) energy, while monitoring reflection coefficients of the surrounding tissue to determine (by inference) the extent of the ablation.

### BACKGROUND

Tissue ablation is a routinely performed procedure that involves heating tissue of various organs, such as the endometrial lining of the uterus, to high temperatures that result in changing the property of cells in the tissue. The changed property may be destruction of cells, coagulation of blood and/or denaturing of tissue proteins. Some currently used methods for ablation include circulation of heated fluid inside the organ, laser treatment of the organ lining, microwave heating of the tissue, high power ultrasound heating of the tissue or resistive heating using application of radiofrequency (RF) energy to the tissue. These ablation procedures, however, are often carried out without direct endoscopic visualization. For example, ablation of the uterine lining or endometrium typically involves insertion of the ablation device into the patient's cervix without the use of a hysteroscope. However, the thickness of the uterine wall may vary from patient-to-patient depending on a number of factors, such as the phase of menstrual cycle, and anatomical variability in the patient. Thus, it is often difficult to determine when the lining of the tissue is sufficiently ablated.

One approach in making this determination involves detecting changes (e.g., impedance) in a transmitter of therapeutic energy, such as an antenna that propagates electromagnetic (i.e., microwave frequency) energy into the surrounding tissue. Typically, efficiency of energy delivery changes when the impedance of any portion of the system changes. Although it is well known to measure impedance of RF systems, it is more difficult with microwave systems because microwave systems do not conduct current to tissue in a circuit for which impedance changes can be easily monitored during the procedure. Most conventionally used methods of determining impedance in microwave systems are inefficient and/or inaccurate due to this challenge.

One prior art approach addressing this problem is described in U.S. Pat. No. 9,526,576 (“Brannan”). In Brannan, a Microwave Research Tool is described that measures impedance in the microwave energy delivery system by measuring broadband scattering parameters at the antenna portion of the device. This measurement may then be used to calibrate the system and to determine subsequent energy delivery. However, this method is deficient in at least that the system fails to take into account a state of the surrounding tissue. Although Brannan utilizes the broadband scattering patterns to determine progress of ablation, Brannan does not disclose any methods related to biophysical feedback received from the tissue. Thus, the system fails to account for individual differences in patients and/or other patient-specific criteria related to ablation.

Similarly, other current methods of monitoring a microwave energy ablation typically rely on time estimations or other generic assumptions to estimate when ablation is complete, and thus are insufficient and/or inefficient and frequently lead to over ablation or under ablation of the tissue lining. This, in turn, has undesirable consequences to the patient, and can often lead to re-emergence of symptoms or even worsening of the condition.

### SUMMARY

In one embodiment of the disclosed inventions, a tissue ablation system includes an antenna, a microwave energy source, measurement apparatus including a signal generator and a detector, a controller, and a switch assembly arranged to alternatively electrically couple the antenna to the microwave energy source or to the measurement apparatus. The controller is configured to cause the switch assembly to alternate between assessment mode and ablation mode, wherein during assessment mode, the switch assembly electrically couples the measurement apparatus to the antenna and the signal generator delivers non-ablative microwave energy to the antenna at a plurality of discrete frequencies and for a duration and power level insufficient to cause thermal injury to tissue proximate to the antenna, and the detector measures a reflection coefficient from the antenna for each discrete frequency of the plurality to thereby obtain a then-current broadband reflection coefficient spectrum of the antenna, and wherein during ablation mode, the switch assembly electrically couples the microwave energy source to the antenna and the microwave energy source delivers ablative microwave energy to the antenna at a selected frequency and for a duration and power level sufficient to cause thermal injury to tissue proximate to the antenna. The controller is further configured to control the delivery of ablative microwave energy during ablation mode (e.g., by modifying one or more of a signal frequency, duration and power level of the ablative microwave energy delivered to the antenna) based at least in part on a difference between a then-current broadband reflection coefficient spectrum and a previously measured broadband reflection coefficient spectrum.

Without limitation, the system may be configured for ablation of endometrial lining tissue of the uterus.

In accordance with one aspect of this embodiment, the controller determines a then-current resonant frequency of the antenna based on a respective then-current broadband reflection coefficient spectrum. The controller may be configured to discontinue delivery of ablative microwave energy when a then-current antenna resonant frequency differs from a prior measured antenna resonant frequency (e.g., an initially measured antenna resonant frequency obtained prior to commencement of any ablation mode) by a predetermined amount. Additionally, and/or alternatively, the controller may determine and take into account a rate of change, a derivative, or an integral based upon successive measured broadband reflection coefficient spectrum curves (i.e., by evaluating changes in the S11 curves over time) for controlling the delivery of ablative microwave energy.

By way of illustration, and without limitation the difference between a then-current broadband reflection coefficient spectrum and a previously measured broadband reflection coefficient spectrum is indicative of one or more changes in characteristics of tissue proximate to the antenna between the respective measurements, such as a depth of ablation in the tissue, a moisture content of the tissue, or an impedance of the tissue.

In another embodiment of the disclosed inventions, a tissue ablation system includes an applicator including a first antenna and a second antenna, a microwave energy source, measurement apparatus including a signal generator and a detector, a controller, and a switch assembly operatively coupled with the respective applicator, microwave energy source, measurement apparatus, and controller. The controller is configured to selectively cause the switch assembly to assume a first switching configuration in which the first antenna is electrically coupled to the microwave energy source and the second antenna is electrically coupled to the measurement apparatus, and to selectively cause the switch assembly to assume a second switching configuration in which the second antenna is electrically coupled to the microwave energy source and the first antenna is electrically coupled to the measurement apparatus. In particular, when the switch assembly is in the first switching configuration, the second antenna is in an assessment mode and the first antenna is in an ablation mode, and wherein when the switch assembly is in the second switching configuration, the first antenna is in an assessment mode and the second antenna is in an ablation mode.

During an assessment mode, the signal generator delivers non-ablative microwave energy to the respective first or second antenna at a plurality of discrete frequencies and for a duration and power level insufficient to cause thermal injury to tissue proximate therewith, and the detector measures a reflection coefficient from said respective first or second antenna for each discrete frequency of the plurality to thereby obtain a then-current broadband reflection coefficient spectrum thereof. During ablation mode, the microwave energy source delivers ablative microwave energy to said respective first or second antenna at a selected frequency and for a duration and power level sufficient to cause thermal injury to tissue proximate therewith. The controller is configured to control the delivery of ablative microwave energy during ablation mode (e.g., by modifying one or more of a signal frequency, duration and power level of the ablative microwave energy delivered to the respective first or second antenna) based at least in part on a difference between a then-current broadband reflection coefficient spectrum and a previously measured broadband reflection coefficient spectrum of one or both of the first and second switches. Without limitation, the system may be configured for ablation of endometrial lining tissue of the uterus.

Without limitation, the controller is preferably configured to obtain an initial broadband reflection coefficient spectrum for each of the first and second antennas before initiating delivery of microwave energy from the microwave energy source in an ablation mode. In accordance with one aspect of this embodiment, the controller is configured to determine a then-current resonant frequency of the first or second antenna based on a then-current broadband reflection coefficient spectrum. The controller may (optionally) be further configured to discontinue delivery of ablative microwave energy to the respective antenna when a then-current antenna resonant frequency differs from a prior measured antenna resonant frequency by a predetermined amount. Without limitation, the prior measured antenna resonant frequency may be an initially measured antenna resonant frequency obtained prior to commencement of any ablation mode.

By way of illustration and without limitation, the difference between a then-current broadband reflection coefficient spectrum and a previously measured broadband reflection coefficient spectrum is indicative of one or more changes in characteristics of tissue proximate to the antenna between the respective measurements, wherein the characteristics of the tissue include a depth of ablation in the tissue, a moisture content of the tissue, and an impedance of the tissue.

In yet another embodiment of the disclosed inventions, a method of ablating tissue, such as uterine endometrial lining tissue, includes positioning an antenna proximate tissue to be ablated; delivering non-ablative microwave energy to the tissue via the antenna at a plurality of discrete frequencies and for a duration and power level insufficient to cause thermal injury to the tissue; measuring a reflection coefficient from the antenna for each discrete frequency of the plurality to thereby obtain a first broadband reflection coefficient spectrum of the antenna; after obtaining the first broadband reflection coefficient spectrum, delivering ablative microwave energy to the tissue via the antenna at a selected frequency and for a duration and power level sufficient to cause thermal injury to the tissue; after delivering ablative microwave energy to the tissue, delivering additional non-ablative microwave energy to the tissue via the antenna at a plurality of discrete frequencies and for a duration and power level insufficient to cause thermal injury to the tissue, and measuring a reflection coefficient from the antenna for each discrete frequency of the plurality to thereby obtain a second broadband reflection coefficient spectrum of the antenna; and after obtaining the second broadband reflection coefficient spectrum, delivering additional ablative microwave energy to the tissue via the antenna at a selected frequency and for a duration and power level sufficient to cause thermal injury to the tissue, including controlling the delivery of additional ablative microwave energy (e.g., by modifying one or more of a signal frequency, duration and power level of the additional ablative microwave energy delivered to the antenna) based at least in part on a difference between the first broadband reflection coefficient spectrum and the second broadband reflection coefficient spectrum.

In accordance with this method, the controller may be configured to determine a first resonant frequency of the antenna based on the first broadband reflection coefficient spectrum, and a second resonant frequency of the antenna based on the second broadband reflection coefficient spectrum, wherein the method may further include discontinuing delivery of ablative microwave energy to the tissue if the second antenna resonant frequency differs from the first antenna resonant frequency by a predetermined amount. For example, the first antenna resonant frequency may be an initial antenna resonant frequency obtained prior to delivery of ablative microwave energy to the tissue.

In one variation of this embodiment, wherein the antenna comprises a first antenna and a second antenna, wherein obtaining the first broadband reflection coefficient spectrum comprises obtaining a respective first broadband reflection coefficient spectrum for each of the first antenna and the second antenna, and wherein obtaining the second broadband reflection coefficient spectrum comprises obtaining a respective second broadband reflection coefficient spectrum for each of the first antenna and the second antenna. For example, the additional non-ablative microwave energy may be delivered to the first antenna for obtaining the second broadband reflection coefficient spectrum while ablative microwave energy is being delivered to the tissue via the second antenna.

These and other aspects and embodiments of the disclosed inventions are described in more detail below, in conjunction with the accompanying figures.

### BRIEF DESCRIPTION OF THE FIGURES

The drawings illustrate the design and utility of embodiments of the disclosed inventions, in which similar elements are referred to by common reference numerals. These drawings are not necessarily drawn to scale. In order to better appreciate how the above-recited and other advantages and objects are obtained, a more particular description of the embodiments will be rendered, which are illustrated in the accompanying drawings. These drawings depict only typical embodiments of the disclosed inventions and are not therefore to be considered limiting of its scope.

FIG. 1 is a combined schematic-block diagram illustrating an exemplary tissue ablation system being used to perform a uterine endometrial lining tissue ablation procedure, in accordance with embodiments of the disclosed inventions;

FIG. 2 is a more detailed schematic diagram illustrating additional details of the tissue ablation system of FIG. 1;

FIGS. 3A-3C illustrate a series of broadband reflection coefficient spectrum curves obtained using the tissue ablation system of FIG. 1;

FIGS. 4A-4C illustrate exemplary flow diagrams depicting various steps for using the tissue ablation system of FIG. 1 to perform a tissue ablation procedure; and

FIGS. 5A-5F are combined schematic-block diagrams depicting a uterine endometrial lining tissue ablation procedure being performed using the tissue ablation system of FIG. 1.

### DETAILED DESCRIPTION OF THE ILLUSTRATED EMBODIMENTS

All numeric values are herein assumed to be modified by the terms “about” or “approximately,” whether or not explicitly indicated, wherein the terms “about” and “approximately” generally refer to a range of numbers that one of skill in the art would consider equivalent to the recited value (i.e., having the same function or result). In some instances, the terms “about” and “approximately” may include numbers that are rounded to the nearest significant figure. The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. In describing the depicted embodiments of the disclosed inventions illustrated in the accompanying figures, specific terminology is employed for the sake of clarity and ease of description. However, the disclosure of this patent specification is not intended to be limited to the specific terminology so selected, and it is to be understood that each specific element includes all technical equivalents that operate in a similar manner. It is to be further understood that the various elements and/or features of different illustrative embodiments may be combined with each other and/or substituted for each other wherever possible within the scope of this disclosure and the appended claims.

Various embodiments of the disclosed inventions are described hereinafter with reference to the figures. It should be noted that the figures are not drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the disclosed inventions, which is defined only by the appended claims and their equivalents. In addition, an illustrated embodiment of the disclosed inventions needs not have all the aspects or advantages shown. For example, an aspect or an advantage described in conjunction with a particular embodiment of the disclosed inventions is not necessarily limited to that embodiment and can be practiced in any other embodiments even if not so illustrated.

Certain types of conditions require the destruction of one or more layers of the inner lining of various body organs. This may be necessary for the treatment or prevention of certain diseases or other physical conditions. For example, dysfunctional uterine bleeding may be such a condition for some women. A common procedure to treat this condition is ablation of the endometrial tissue layer (or “lining”) of the uterus. Ablation of tissue involves delivering energy to the tissue so as to generate heat and cause tissue necrosis without necessarily contacting the tissue. The ablation achieves destruction of cells in the lining of the tissue, thereby changing some properties of the tissue. For purposes of illustration, the tissue ablation system and methods of use are disclosed and described herein in the context of performing endometrial lining tissue ablations. However, the invention(s) described herein may similarly be used for tissue ablation procedures conducted in other body cavities, organs or solid tissue in which electromagnetic microwave energy may be effectively used, and the current disclosure should not be read as limiting the invention(s) to endometrial tissue ablation procedures.

The current disclosure describes an ablation system that switches between an “ablation mode” and an “assessment mode” in order to deliver an optimal amount of energy sufficient to change one or more properties of the tissue and, more specifically, to periodically monitor and measure properties of the surrounding tissue in order to determine if the ablation is sufficient to achieve the therapeutic goal. In particular, rather than relying on a standard or generic measure that is indicative of optimal ablation on average (e.g., a certain amount of time, a certain amount of energy, etc.), the disclosed and described ablation system periodically receives and analyzes bio-physical feedback to determine when ablation is optimal/sufficient for a particular patient and/or tissue type.

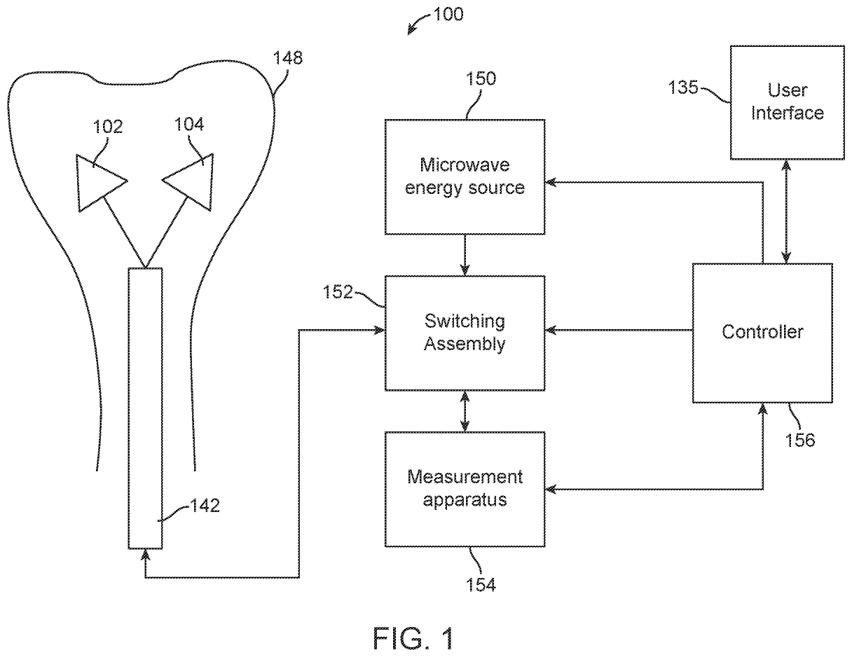

Referring now to FIG. 1, in accordance with the present disclosure, a microwave energy ablation system 100 is constructed for performing tissue ablation procedures on a mammalian (e.g., human female) patient. The system includes an insertable portion comprising an applicator body 142 and a pair of antennas 102 and 104. In FIG. 1, the insertable portion of the system 100 is shown inserted in a body cavity 148 for ablating lining (wall) tissue within the cavity. As discussed above, the cavity 148 may be any body cavity, and the system 100 is illustrated and described while performing a uterine endometrial lining tissue ablation procedure for purposes of illustration, and not limitation. It should also be appreciated that although the illustrated system 100 employs two antennas 102 and 104, alternative embodiments of the system 100 may utilize a single antenna, or possible three or more antennas.

The applicator 142 may be the housing for electrical connections (e.g., supply lines and return lines) that power the antennas 102 and 104. The antennas 102 and 104 may be electro-thermal elements that are operatively coupled to one or more supply lines of the cables housed in the applicator 142. The antennas 102 and 104 are configured to deliver electromagnetic (hereinafter “microwave”) energy to the body cavity 148 in both a narrowband range and a broadband range, as will be discussed in further detail below. The antennas 102 and 104 are preferably selected and configured to yield resonance at a system operating frequency, and each preferably has an adjustable shape in order to conform to the anatomy of a given patient. Thus, the antennas 102 and 104 should be designed keeping such a functionality in mind. For example, various parameters of the antennas 102 and 104, e.g., shape, length, radiating elements, etc., may be modified based on the desired transmission frequency range. Further details on devices and methods for delivering microwave energy are disclosed in U.S. patent application Ser. No. 15/256,259, entitled “Returned Power for Microwave applications,” which is herein incorporated by reference in its entirety.

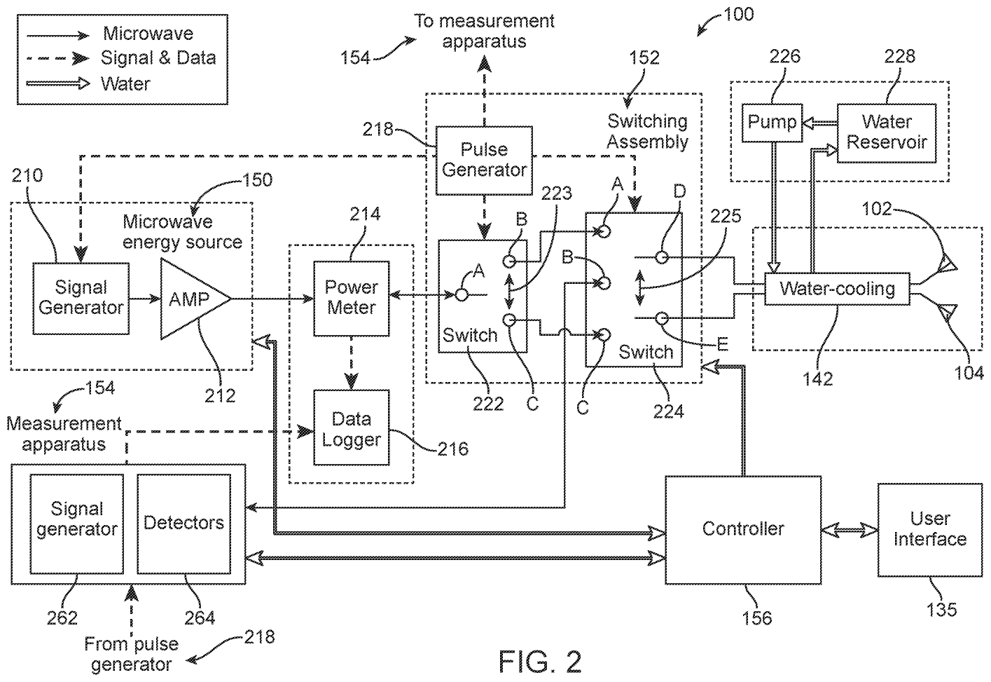

The ablation system 100 includes a microwave energy source 150 that provides narrowband microwave ablation energy that is delivered to the antennas 102 and 104 through the applicator 142. As described below in greater detail with respect to FIG. 2, the microwave energy source 150 includes a signal generator that is capable of generating and supplying a high frequency microwave signal, and an amplifier for amplifying the high frequency microwave signal to a suitable tissue ablation power. In various embodiments, the microwave energy source 150 may deliver a signal at an operating frequency within a range of around 850 MHz to around 1.2 GHz. However, it should be appreciated that the operating frequency may be fixed for a particular patient or procedure. For example, for some patients and procedures, the microwave energy source 150 may output a signal at or about 915 MHz. The microwave energy source 150 preferably includes one or more active output terminals (not shown) that deliver energy to the antennas via one or more electrical cables and/or other connections.

The ablation system 100 also includes a measurement apparatus 154 that is configured to calculate broadband reflection coefficients associated with the ablated tissue. As described below in greater detail with respect to FIG. 2, the measurement apparatus 154 includes a multifrequency signal generator that is capable of generating and supplying high frequency microwave signals and one or more detectors configured to measure forward and reflected power. As will be further discussed below, during a tissue ablation procedure, it is anticipated that there will be shifts and/or changes to a shape of a reflection coefficient spectrum. These changes in the broadband reflection coefficient spectrum, referred in the art as the “S11 spectrum,” may be monitored to infer changes in the properties of the surrounding tissue, e.g., degree of ablation, depth of ablation, area of ablation, moisture content, etc.). The changes to the S11 spectrum may then be used to assess ablation progress.

The ablation system 100 further includes a switching assembly 152 comprising one or more positioning switches that are configured to alternatively electrically couple the applicator 142 and antennas 102 and 104 to the microwave energy source 150, the measurement apparatus 154, or both. A system controller 156 is operatively coupled with, and configured to control each of, the microwave energy source 150, the measurement apparatus 154, and the switching assembly 152. A user interface 135 is operatively coupled with the controller and configured for displaying system status and/or inquires to a system operator, and for receiving user inputs in response to the displayed system status and/or inquires.

In particular, the controller 156 is configured to cause the switching assembly 152 to selectively couple the measurement apparatus 154 to one or both antennas 102 and 104, and to initiate delivery of non-ablative microwave energy from the measurement apparatus 154 via the respective antennas 102 and 104, to the surrounding tissue within cavity 148, which may be (without limitation) the endometrial lining tissue of a uterus, as part of an “assessment mode” in which the microwave energy is delivered at a plurality of discrete frequencies and for a time and intensity insufficient to cause any significant heating of the tissue. The measurement apparatus 154 is configured to measure a reflection coefficient for each discrete frequency to obtain an initial broadband reflection coefficient spectrum representative of the tissue. The controller 156 then causes the switch assembly 152 to electrically couple the microwave ablation energy source 150 to the antennas 102 and 104 for delivery of microwave ablation energy to the tissue in an “ablation mode” in which microwave energy is delivered at a predetermined frequency and for a time and intensity sufficient to cause ablation of the tissue. It should be appreciated that alternative embodiments may utilize only a single antenna that is alternatively switched between an ablation mode and an assessment mode.

As will be further described herein, the controller 156 periodically causes the switching assembly 152 to alternate between the ablation mode and the assessment mode, wherein during each assessment mode a current broadband reflection coefficient spectrum measurement is obtained. In particular, the controller 156 is configured to control the delivery of microwave energy from the microwave energy source 150 to the patient cavity 148 based at least in part on a difference between the initial broadband reflection coefficient spectrum and the current broadband reflection coefficient spectrum obtained during each assessment mode. Towards this end, and as described in greater detail herein, in addition to performing other functions, the controller 156 analyzes and compares data received from the measurement apparatus 154 to a predetermined range of S11 spectrum profiles that are indicative of adequate ablation.

FIG. 2 includes a more detailed schematic diagram of the microwave ablation system 100, depicting the various components and subcomponents thereof.

The microwave energy source 150 comprises a signal generator 210 that is configured to generate a narrowband microwave signal at a predetermined frequency (e.g., 850 MHz, 915 MHz, etc.). The signal generator 210 is coupled to an amplifier 212 that amplifies the signal level to a range required (e.g., 10-100 W) for tissue ablation, under the control of the controller 156. In one or more embodiments, the signal generator 210 may be a high-power microwave source, such as a magnetron. In various embodiments of the system 100, the signal generator 210 may have a variable signal frequency, which is controlled by the controller 156. For example, different narrowband frequencies may be selected by the controller 156 depending on the particular patient and/or anatomy to be ablated, limited procedure time, etc.

The output of the microwave energy source 150 is coupled to a first node A of a first switch 222 of the switching assembly 152 via a power meter 214. The switch 222 selectively connects node A with one of nodes B and C (or neither), as indicated by the arrow 223. The power meter 214 is configured to measure both forward and reflected power levels between the microwave power source 150 and antennas 102 and/or 104 via switch 222 (as explained below). In the illustrated embodiment, the power level measured by the power meter 214 is stored in a data logger 216 that records the ablation procedure data for collecting data of multiple procedures and general data collection purposes. In some embodiments, the measured power levels from the power meter 214 may also be used as feedback provided to the controller 156, e.g., for controlling the amplifier 212.

The measurement apparatus 154 includes a broadband microwave signal generator 262, and one or more reflected signal detectors 264. Without limitation, the signal generating and detecting/measurement functions of the measurement apparatus 154 may be performed by a vector network analyzer (VNA) that generates signals at multiple frequencies and measures amplitude and/or phase of both forward and reflected signals. This information is used to calculate reflection coefficients (as explained below). In particular, the power sensors/detectors 264 are selectively coupled to the antennas 102 and 104 via a second switch 224 of the switching assembly 152 in order to measure the power reflected back from the tissue due to mismatches between the feeding transmission line and the antennas. As explained in greater detail herein, the measured reflected power is used by the measurement apparatus 154 to calculate reflection coefficients based on impedance measurements measured at the body cavity in order to assess ablation progress and/or determine whether optimal ablation has been achieved.

More specifically, the measure apparatus 154 is electrically connected to node B of switch 224 of the switching assembly 152, which selective connects node B with one of nodes D and E (or neither), as indicated by the arrow 225. In this manner, the broadband signal generator 262 may be selectively electrically coupled by switch 224 with a respective one of antennas 102 and 104. When coupled to a respective antenna 102/104, the signal generator 262 generates and transmits a broadband signal (spanning a wide range of frequencies) for a time and intensity insufficient to cause any meaningful (i.e., injury inducing) heating of the tissue. The one or more detectors 264 are configured to measure both the forward and reflected power to/from the antenna(s) 102/104, similar to the power meter 214. In particular, the detectors 264 are configured to measure an impedance mismatch between a transmission line (i.e., cable) and the antenna(s) 102/104. Since the antennas' impedance is influenced by the electrical properties of the material surrounding the antenna, this provides an assessment of the electrical or physical state of the patient's tissue in proximity to the respective antenna 102/104. The measured data is used by the measurement apparatus 154 to calculate the reflection coefficients at each of the discrete frequencies and may also be recorded by data logger 216 as part of the stored ablation procedure data.

The switching assembly 152 generally comprises a pulse generator 218 and respective positional (e.g., selector) switches 222 and 224, which are collectively under control of the controller 156. The pulse generator 218 generates the signal for controlling the respective switches 222/224 and also enables/disables signal generator 210 of the microwave power source 150, and signal generator 262 of the measurement apparatus 154, respectively. For example, when the system 100 is in an assessment mode, the pulse generator 218 may disable the output of the microwave source 150 while near-simultaneously enabling the output of the measurement apparatus 154. It should be appreciated that, in alternative embodiments, the pulse generator signals for controlling the timing and operation of the respective system components may alternatively be supplied by the controller 156, or some other module that is distinct from the switching assembly 152. What matters is that the timing signals and operational pulses supplied to the respective system components 150, 152, 154, 156 are synchronized.

The first positional switch 222 is configured to directly electrically connect the narrowband microwave energy source 150 (via the power meter 214) to either antenna 102 via node B, or antenna 104 via node C, in each case additionally based on the position of the second switch 224. The switch 222 can also be in a neutral position, as shown in FIG. 2, in which neither node B or C is connected to node A. Movement of the switch 222 between connecting nodes A-B, neutral, or nodes A-C, respectively, is indicated by arrow 223.

The second positional switch 224 is configured to selectively switch between a first position in which node A of switch 224 (microwave energy generator 150 via node B of switch 222) is electrically connected to node D (antenna 102) and node B of switch 224 (measurement apparatus 154) is electrically connected to node E (antenna 104), and a second position in which node B of switch 224 (measurement apparatus 154) is electrically connected to node D (antenna 102) and node C of switch 224 (microwave energy generator 150 via node C of switch 222) is electrically connected to node E (antenna 104). The switch 224 can also be in a neutral position in which none of nodes A, B or C are connected to nodes D or E, as shown in FIG. 2. Movement of the switch 224 between the first position connecting nodes A-D and B-E, neutral, and the second position connecting nodes B-D and C-E, respectively, is indicated by arrow 225. Alternate embodiments of the ablation system 100 may employ only a single switching device capable of switching between the microwave energy source 150 and the measurement apparatus 154. Similarly, more than two switches may be envisioned for other alternative embodiments that have more components (e.g., antennas). For example, if an alternative embodiment of the ablation system 100 comprises three or four antennas, three or four (or more) switches may be used.

The ablation system 200 includes an optional coolant circulation system comprising a pump 226 and a coolant reservoir 228 to maintain the applicator 142 and antennas 102/104 at safe temperature levels. In particular, the pump 226 and the reservoir 228 circulate the coolant through the applicator 142. The coolant circulation system may include a temperature regulation system (not shown) to control the temperature of the circulation coolant, which may be water or saline, although any suitable coolant may be used instead. Other liquids or gases having the appropriate heat capacity, thermal conductivity and viscosity may be similarly substituted. A passive cooling system may alternatively be employed, such as use of an air gap or thermally insulative material in the applicator. In any event, the applicator 142 is configured to be inserted into the body cavity houses a set of electrical connections that deliver energy to the antennas 102 and 104. As discussed above, some embodiments may have a single antenna, whereas others (included the illustrated embodiment) may have two antennas 102 and 104.

As discussed above in conjunction with FIG. 1, the controller 156 is operatively coupled with the microwave source 150, the switching assembly 152, the measurement apparatus 154, and the power meter 214, and controls the operation of the ablation system 100. In particular, the controller 156 comprises processing circuitry that controls logic for the functioning of the various components of the ablation system 100. The controller 156 may be implemented as software on a microcomputer or other processing device, and processes and provides one or more signals sent to and received from the various hardware components of the ablation system 100 (e.g., microwave energy source 150, measure apparatus 154, switching assembly 152, etc.). The controller 156 provides or otherwise regulates the timing logic for the ablation system 100, for example, to determine a duration of delivering microwave energy to the tissue. The controller 156 includes the respective circuitry, logic and memory necessary to determine when to switch between ablation mode and assessment mode, and more particularly to determine, based on the broadband reflection coefficient spectrum, whether a current S11 spectrum indicates that optimal ablation has been achieved. In one or more embodiments, the controller 156 may consult a library or database to compare a set of data associated with the most recent S11 spectrum with a data set associated with an optimized S11 spectrum. If/once the set of data falls within an acceptable/predetermined range of the optimized S11 spectrum, the controller 156 may determine that optimal ablation has been achieved. Additionally or alternatively, it may be advantageous or preferred to not just compare the absolute difference between a current resonant frequency and the initial resonant frequency, but to further (or instead) examine one or more of a rate of change, derivatives, integrals and other trends between the respective broadband reflection coefficient spectrums (i.e., by evaluating changes in the S11 curves over time) as the ablation procedure progresses in order to control the ablation procedure (e.g., by adjusting one or more of a signal frequency, duration and power level of the ablative microwave energy delivered to the antenna(s)) or otherwise determine that optimal ablation has been achieved.

In addition to determining when to terminate the ablation procedure, the controller 156 may also adjust the ablation power level supplied by the microwave energy source 150. For example, depending on data received during assessment, the controller may modify one or more criteria related to the microwave power source 150 (e.g., modify intensity, time, duration, etc.). In some embodiments, the controller 156 may dynamically adjust the switching time between ablation and assessment modes rather than enforcing a predetermined switching interval. In other words, based on data received during an assessment mode, the controller 156 may dynamically modify intervals of ablation. For example, a first ablation interval may be 5 seconds. But a subsequent interval may be reduced to 3 seconds if it is determined that the ablation is near optimal.

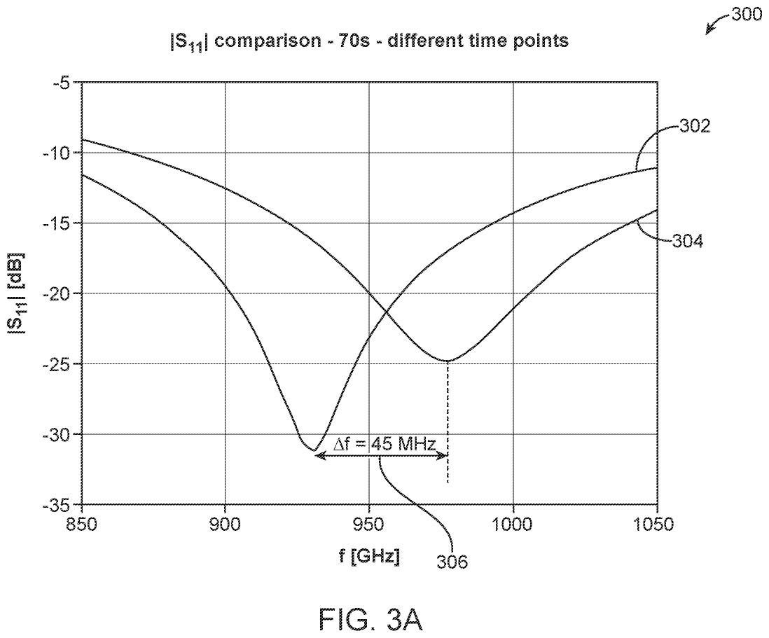

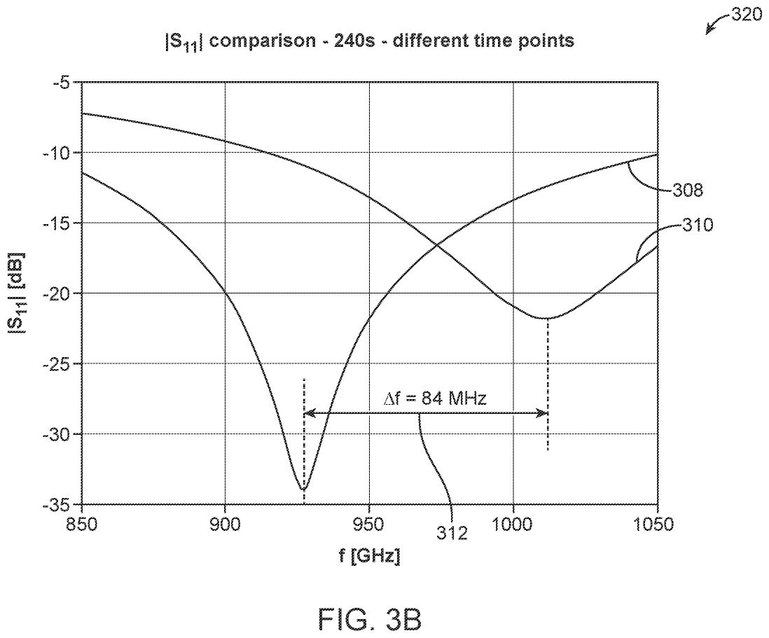

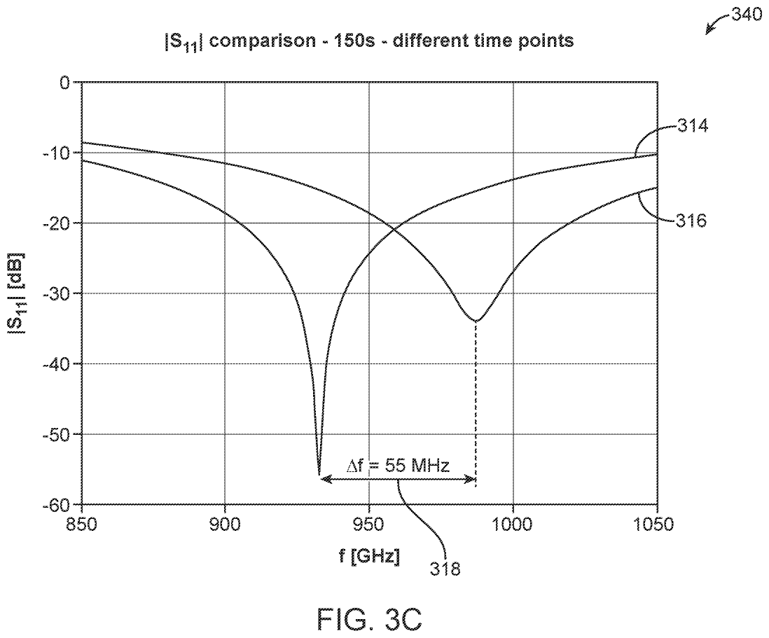

FIGS. 3A-3C illustrate exemplary S11 spectrums indicative of various stages of an ablation procedure. In order to determine the S11 spectrum, when in an assessment mode, the ablation system 100 delivers broadband microwave signal through the broadband signal generator 262 of the measurement apparatus 154 (see FIG. 2). The system 100 then measures reflection coefficients for each discrete frequency to obtain an initial broadband reflection coefficient spectrum (initial S11 spectrum). In particular, as discussed above, the reflection coefficients are measured by the one or more detectors 264 which are configured to measure power reflected back from the tissue. As tissue properties change during the course of an ablation procedure, the reflection coefficients at the various frequencies of the broadband signal also change. This change over time may be advantageously used to determine when the particular ablation procedure is optimally complete.

In general, the scattering, or S-parameters, describe the input-output relationship between the ports of an electrical network. The S11 parameter describes the how much power is reflected from the antenna connected to an electrical transmission line. One approach to determine a difference between an initial S11 spectrum and a current S11 spectrum is to focus on a change in resonant frequency. Resonant frequency is the frequency at which the S11 value is at a minimum. This indicates a minimal amount of reflected power at a particular frequency. It should be noted that the resonant frequency is a function of electrical properties of the tissue medium surrounding the antenna(s). A change in the resonant frequency may indicate that the electrical properties of the surrounding medium has changed.

Therefore, observing changes in the resonant frequency of the S11 spectrum may be a good approach to detect change in the electrical properties of the tissue surrounding the antenna(s).

Accordingly, a pre-ablation S11 spectrum is determined at the start of the procedure, i.e., prior to any ablation energy being delivered. This pre-ablation S11 spectrum data is then stored (e.g., by the controller 156) for comparison at subsequent measurement intervals. After the initial S11 spectrum has been measured, the system 100 operates in the ablation mode and delivers narrowband microwave energy to the tissue around the antennas 102/104 for a predetermined amount of time (e.g., 5 seconds). The system 100 then switches back to an assessment mode and again delivers a broadband signal to the tissue around the antenna(s) 102/104, and a current S11 spectrum is determined. This S11 spectrum is compared to the initial S11 spectrum in order to determine whether ablation is sufficient. If yes, the system 100 terminates the process. If no, the system 100 returns to the ablation mode and delivers more narrowband microwave energy to the tissue for the predetermined amount of time again. As discussed above, some embodiments allow for the ablation parameters (e.g., power level, interval, etc.) to be dynamically adjusted over time. This is repeated until data associated with the most recent S11 spectrum falls within a predetermined range of an optimal S11 spectrum.

FIGS. 3A-3C each illustrate two S11 curves: a pre-ablation S11 curve (302, 308, 314), and a post-ablation S11 curve (304, 310, 316). In FIG. 3A, the Δf 306 (the difference between the peaks of both graphs) is 45 MHz, which is indicative of tissue that is under-ablated. Thus, if Δf is determined to be in this range, the ablation system may determine that an additional round of ablation in ablation mode is necessary. Although the current example discusses determination of optimal ablation based on a Δf determination, any other characteristic of the spectrum curves could be similarly compared to determine optimal ablation. In FIG. 3B, the Δf 312 is 84 MHz. This Δf is indicative of tissue that has been over-ablated. Based on this S11 spectrum, the system may determine that ablation is past the optimal range and terminate the ablation procedure. FIG. 3C illustrates an S11 spectrum curve that is indicative of adequate ablation. The Δf, in this case is 55 MHz. If, after a particular ablation interval, the S11 spectrum, or the associated Δf falls within this range, the ablation system determines that optimal/sufficient ablation has been achieved, and terminates the procedure.

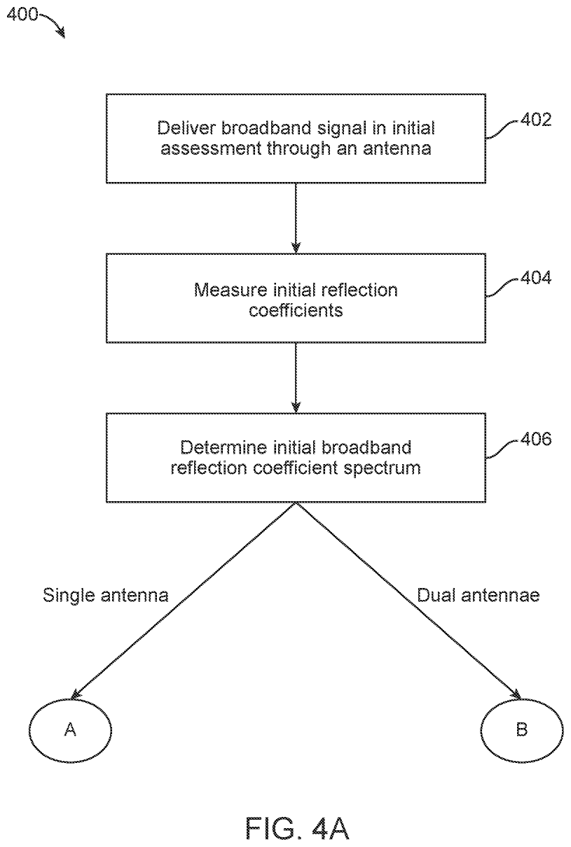

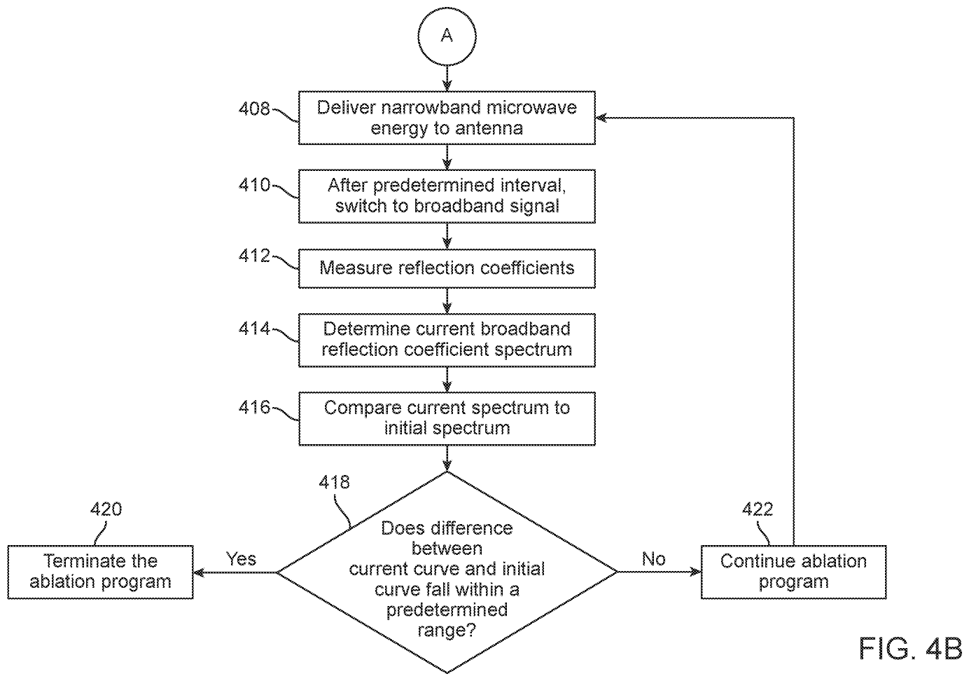

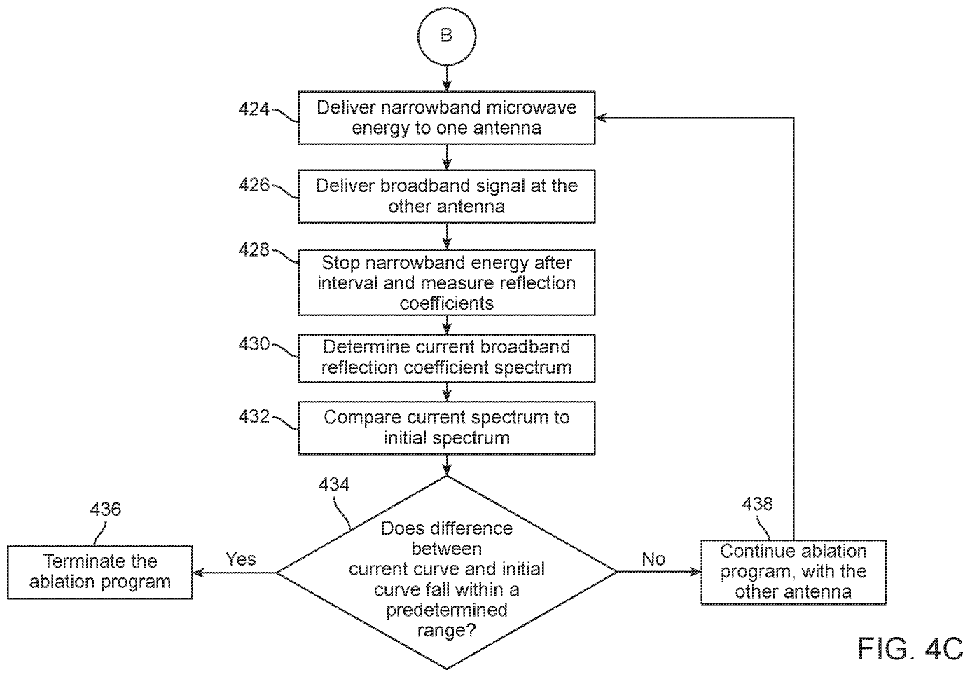

Referring now to FIGS. 4A-4C, an example process flow diagram 400 is illustrated. At 402, the ablation system, in assessment mode, delivers broadband microwave signal to the antenna(s). At 404, the ablation system measures the initial reflection coefficients at each frequency of the broadband microwave signal. At 406, an initial S11 spectrum is determined. This initial S11 spectrum is stored.

If the ablation system comprises a single antenna, at 408, ablation mode is initiated, and narrowband microwave energy is delivered to the single antenna. At 410, after a predetermined interval of ablation (e.g., 5 seconds), the switching assembly 152 switches the single antenna from the microwave signal 150 to the broadband signal generator 262. At 412, in response to the broadband signal delivered to the tissue, reflection coefficients for the range of frequencies is measured. At 414, a current S11 spectrum curve is determined. At 416, the current S11 spectrum is compared to the initial S11 spectrum curve.

If, at 418, it is determined that the difference between the current S11 curve and the initial S11 curve falls within a predetermined range, the ablation program may be terminated at 420. If, at 418, it is instead determined that the difference between the current S11 curve and the initial S11 curve does not fall within the predetermined range (e.g., falls below the range, or a particular number), the ablation program may be continued at 422. If the ablation program is continued, the switching assembly 152 may once again switch the energy source from the broadband signal generator 262 to the microwave power source 150 such that narrowband signals are generated at the single antenna for the predetermined time interval. This process continues until the difference between the S11 curves fall within the acceptable range, and the ablation program is deemed to be complete.

If the ablation system comprises dual antennas, at 424, after the initial assessment (performed by either antenna, or even both antennas), a first antenna delivers narrowband microwave energy to the tissue for a predetermined time interval (e.g., 5 seconds, etc.). Simultaneously, at 426, the second antenna is coupled to the broadband signal generator 262, and operates in an assessment mode. At 428, after the predetermined time interval has elapsed, the narrowband microwave energy from the first antenna is terminated, and reflection coefficients measured at the second antenna are measured, and a current S11 curve is determined. The current S11 curve is compared with the initial S11 curve, at 432. If, at 434, it is determined, based on a comparison of the current S11 curve and the initial S11 curve, that the current S11 curve falls within a predetermined range, the ablation program may be terminated at 436. If, at 434, it is instead determined, based on a comparison of the current S11 curve and the initial S11 curve, that the current S11 curve does not fall within the predetermined range (e.g., falls below the range, or a particular number), the ablation program may be continued from the second antenna, at 438.

The ablation program continues with the second antenna being now coupled to the microwave energy source 150, and the first antenna coupled to the broadband signal generator 262. Thus, the two antennas switch between the ablation mode and the assessment mode until it is determined that the S11 curve falls within the predetermined range that indicates sufficient ablation.

In alternative embodiments, the first antenna may be designated as an “ablation antenna” and the second antenna may be designated as an assessment antenna. In such a case, even after it is determined that the ablation is not sufficient, rather than switching modes between the two antennas (as described in FIG. 4C), each antenna continues with the same function. For example, if the first antenna performed ablation, and the second antenna performed measurement, after a first round of comparison of S11 curve, the first antenna again performs ablation, and the second antenna again performs measurement.

In yet other alternative embodiments, if, based on the comparison of S11 curves, it is determined that ablation is not sufficient (but close to being sufficient, perhaps), an intensity or interval of microwave energy may be suitably modified. For example, if, after a few rounds of ablation, the S11 curve indicates that ablation is close to being sufficient, one or more parameters (e.g., time, intensity, frequency, power level, etc.) of the narrowband microwave energy may be modified for the next round.

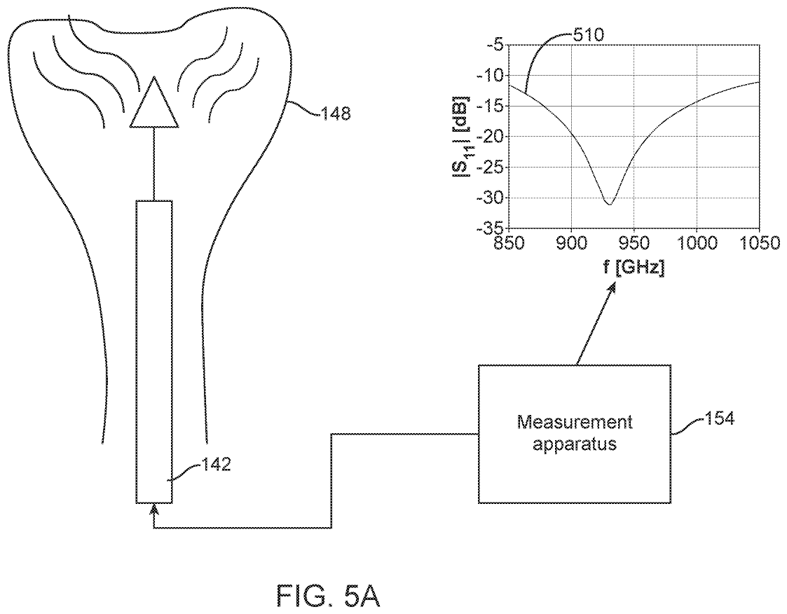

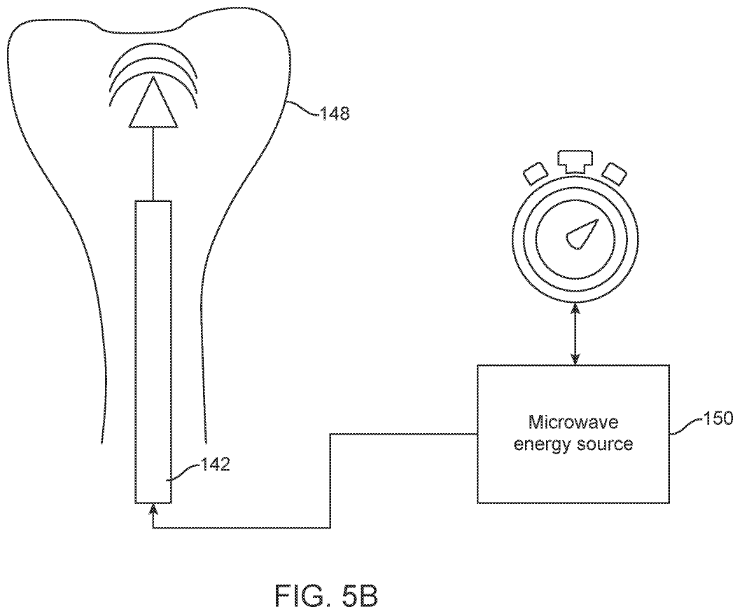

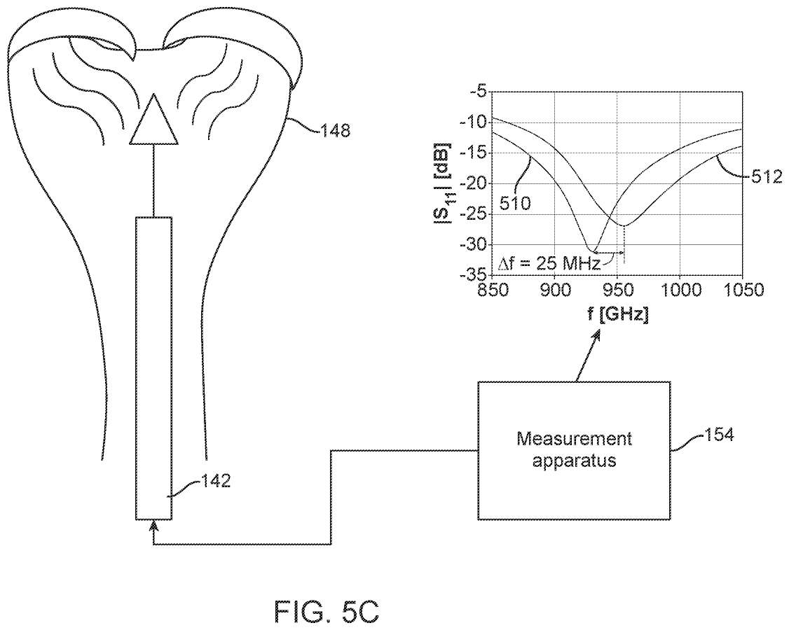

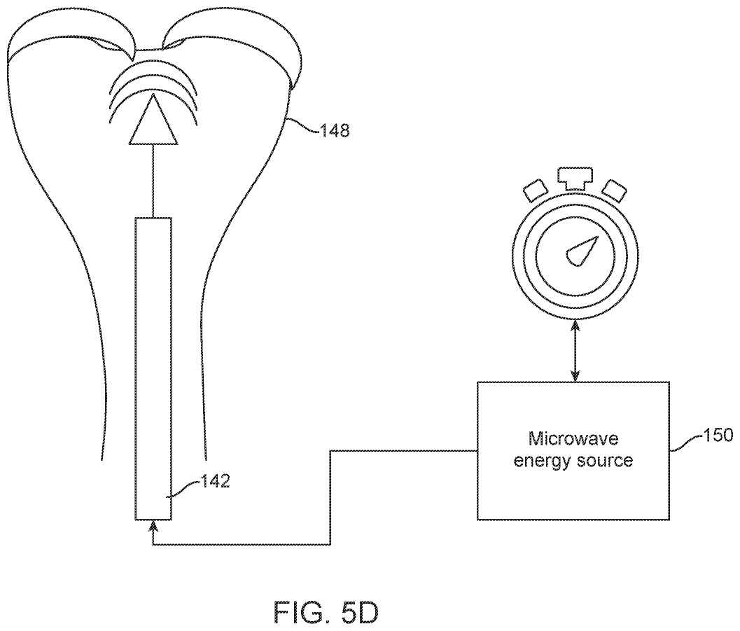

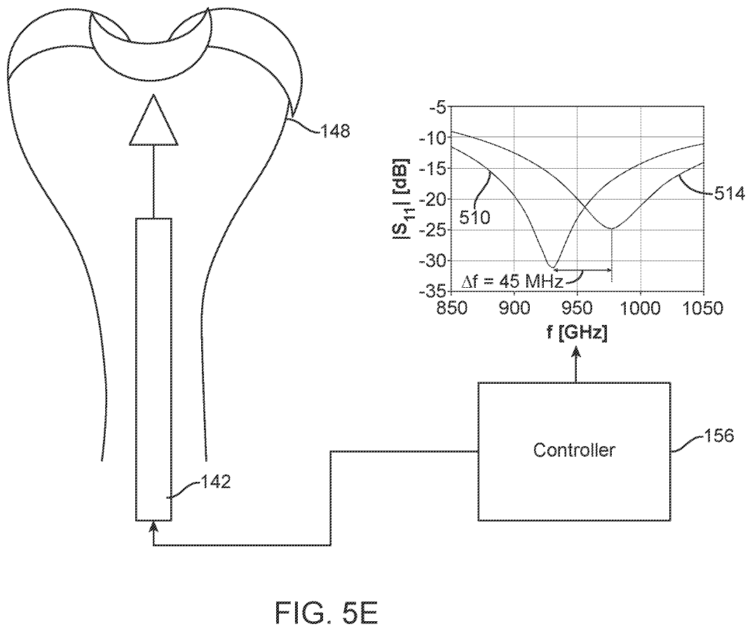

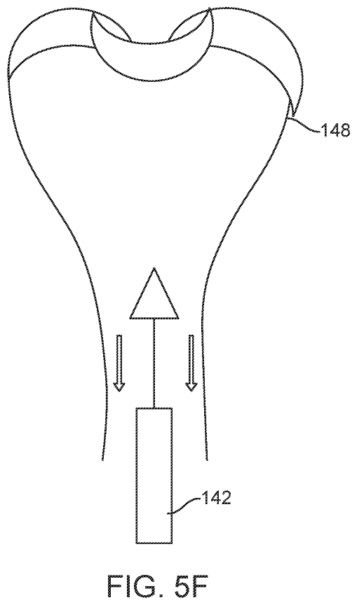

Referring now to process flow diagrams shown in FIGS. 5A-5F, FIG. 5A illustrates an initial assessment of the tissue using an ablation system applicator 142 having a single antenna to ablate tissue in a body cavity (e.g., uterine cavity) 148. In the initial assessment, broadband signal is delivered to the tissue through the measurement apparatus 154. An initial S11 curve 510 is determined based on the determined reflected coefficients.

As shown in FIG. 5B, the switching assembly 152 decouples the measurement apparatus 154 from the single antenna and couples the microwave energy source 150 that delivers narrowband microwave energy to the surrounding tissue in the body cavity 148 for a predetermined time interval (ablation mode).

As shown in FIG. 5C, the switching assembly 152 decouples the microwave energy source 150 from the single antenna and couples the measurement apparatus 154 to the single antenna (“assessment mode”). As shown in FIG. 5C, one or more properties of the tissue in cavity 148 may have changed (as indicated by “hardened tissue”) based on the ablation. Another S11 curve 512 based on the current reflection coefficients is determined, and the initial S11 curve 510 may be compared to the current S11 curve 512. It may be determined that ablation is not sufficient based on the difference between curves 511 and 512.

As shown in FIG. 5D, the switching assembly 152 once again switches back to ablation mode for the predetermined interval.

As shown in FIG. 5E, the switching assembly 152 switches back to assessment mode, and the controller 156 compares the most recent S11 curve 514 to the initial S11 curve 510. It may be determined that the two curves fall within an acceptable range of one another to indicate that the ablation was sufficient.

As shown in FIG. 5F, since ablation has been deemed complete, the applicator 142 is removed from the body cavity.

It should be further appreciated that the use of a passive element coupled with the antennas may be beneficial, as is described and explained in the appended document authored by one (or more) of the inventors, the contents of which are to be considered as part of the present disclosure.

Having described exemplary embodiments of the disclosed microwave tissue ablation system, it can be appreciated that the examples described above and depicted in the accompanying figures are only illustrative, and that other embodiments and examples also are encompassed within the scope of the appended claims. For example, while the flow diagrams provided in the accompanying figures are illustrative of exemplary steps; the overall image merge process may be achieved in a variety of manners using other data merge methods known in the art. The system block diagrams are similarly representative only, illustrating functional delineations that are not to be viewed as limiting requirements of the disclosed inventions. It will also be apparent to those skilled in the art that various changes and modifications may be made to the depicted and/or described embodiments (e.g., the dimensions of various parts), without departing from the scope of the disclosed inventions, which is to be defined only by the following claims and their equivalents. The specification and drawings are, accordingly, to be regarded in an illustrative rather than restrictive sense.

1. A tissue ablation system, comprising:
an antenna;
a microwave energy source;
measurement apparatus including a signal generator and a detector;
a controller; and
a switch assembly arranged to alternatively electrically couple the antenna to the microwave energy source or to the measurement apparatus,
wherein the controller is configured to cause the switch assembly to alternate between assessment mode and ablation mode,
wherein during assessment mode, the switch assembly electrically couples the measurement apparatus to the antenna and the signal generator delivers non-ablative microwave energy to the antenna at a plurality of discrete frequencies and for a duration and power level insufficient to cause thermal injury to tissue proximate to the antenna, and the detector measures a reflection coefficient from the antenna for each discrete frequency of the plurality to thereby obtain a then-current broadband reflection coefficient spectrum of the antenna, and
wherein during ablation mode, the switch assembly electrically couples the microwave energy source to the antenna and the microwave energy source delivers ablative microwave energy to the antenna at a selected frequency and for a duration and power level sufficient to cause thermal injury to tissue proximate to the antenna, and
wherein the controller is configured to control the delivery of ablative microwave energy during ablation mode based at least in part on a difference between a then-current broadband reflection coefficient spectrum and a previously measured broadband reflection coefficient spectrum.

2. The tissue ablation system of claim 1, wherein the system is configured for treating endometrial lining tissue of the uterus.

3. The tissue ablation system of claim 1, wherein controlling the delivery of ablative microwave energy includes modifying one or more of a signal frequency, duration and power level of the ablative microwave energy delivered to the antenna.

4. The tissue ablation system of claim 1, wherein the controller determines and takes into account a rate of change, a derivative, or an integral based upon successive measured broadband reflection coefficient spectrums for controlling the delivery of ablative microwave energy.

5. The tissue ablation system of claim 1, wherein the controller is configured to determine a then-current resonant frequency of the antenna based on a respective then-current broadband reflection coefficient spectrum of the antenna, and to discontinue delivery of ablative microwave energy when a then-current antenna resonant frequency differs from a prior measured antenna resonant frequency by a predetermined amount.

6. The tissue ablation system of claim 5, wherein the prior measured antenna resonant frequency is an initially measured antenna resonant frequency obtained prior to commencement of any ablation mode.

7. The tissue ablation system of claim 1, wherein the difference between a then-current broadband reflection coefficient spectrum and a previously measured broadband reflection coefficient spectrum is indicative of one or more changes in characteristics of tissue proximate to the antenna between the respective measurements.

8. The tissue ablation system of claim 7, wherein the characteristics of the tissue include one or more of a depth of ablation in the tissue, a moisture content of the tissue, and an impedance of the tissue.

9. A tissue ablation system, comprising:
an applicator including a first antenna and a second antenna;
a microwave energy source;
measurement apparatus including a signal generator and a detector;
a controller; and
a switch assembly operatively coupled with the respective applicator, microwave energy source, measurement apparatus, and controller,
wherein the controller is configured to selectively cause the switch assembly to assume a first switching configuration in which the first antenna is electrically coupled to the microwave energy source and the second antenna is electrically coupled to the measurement apparatus, and to selectively cause the switch assembly to assume a second switching configuration in which the second antenna is electrically coupled to the microwave energy source and the first antenna is electrically coupled to the measurement apparatus,
wherein when the switch assembly is in the first switching configuration, the second antenna is in an assessment mode and the first antenna is in an ablation mode, and wherein when the switch assembly is in the second switching configuration, the first antenna is in an assessment mode and the second antenna is in an ablation mode,
wherein during an assessment mode, the signal generator delivers non-ablative microwave energy to the respective first or second antenna at a plurality of discrete frequencies and for a duration and power level insufficient to cause thermal injury to tissue proximate therewith, and the detector measures a reflection coefficient from said respective first or second antenna for each discrete frequency of the plurality to thereby obtain a then-current broadband reflection coefficient spectrum thereof,
wherein during ablation mode, the microwave energy source delivers ablative microwave energy to said respective first or second antenna at a selected frequency and for a duration and power level sufficient to cause thermal injury to tissue proximate therewith, and
wherein the controller is configured to control the delivery of ablative microwave energy during ablation mode based at least in part on a difference between a then-current broadband reflection coefficient spectrum and a previously measured broadband reflection coefficient spectrum of one or both of the first and second switches.

10. The tissue ablation system of claim 9, wherein the controller is configured to obtain an initial broadband reflection coefficient spectrum for each of the first and second antennas before initiating delivery of microwave energy from the microwave energy source in an ablation mode.

11. The tissue ablation system of claim 9, wherein the system is configured for treating endometrial lining tissue of the uterus.

12. The tissue ablation system of claim 9, wherein controlling the delivery of ablative microwave energy during an ablation mode includes modifying one or more of a signal frequency, duration and power level of the ablative microwave energy delivered to the respective first or second antenna.

13. The tissue ablation system of claim 9, wherein the controller is configured to determine a then-current resonant frequency of the first or second antenna based on a then-current broadband reflection coefficient spectrum of the respective antenna, and to discontinue delivery of ablative microwave energy to the respective antenna when a then-current antenna resonant frequency differs from a prior measured antenna resonant frequency by a predetermined amount.

14. The tissue ablation system of claim 13, wherein the prior measured antenna resonant frequency is an initially measured antenna resonant frequency obtained prior to commencement of any ablation mode.

15. The tissue ablation system of claim 9, wherein the difference between a then-current broadband reflection coefficient spectrum and a previously measured broadband reflection coefficient spectrum is indicative of one or more changes in characteristics of tissue proximate to the antenna between the respective measurements, wherein the characteristics of the tissue include a depth of ablation in the tissue, a moisture content of the tissue, and an impedance of the tissue.

16-23. (canceled)
